(19) United States
(12) Patent Application Publication
(10) Pub. No.: US 2016/0000340 A1
(43) Pub. Date: Jan. 7, 2016

(54) RAPID CENTRAL VENOUS PRESSURE MONITOR

(71) Applicants: Michael V. Jackson, Reno, NV (US); Raymond D. Evans, Reno, NV (US); Donald L. Evans, Reno, NV (US); Timothy A. Rosemore, Reno, NV (US)

(72) Inventors: Michael V. Jackson, Reno, NV (US); Raymond D. Evans, Reno, NV (US); Donald L. Evans, Reno, NV (US); Timothy A. Rosemore, Reno, NV (US)

(21) Appl. No.: 14/792,186

(22) Filed: Jul. 6, 2015

Related U.S. Application Data
(60) Provisional application No. 62/020,594, filed on Jul. 3, 2014.

(51) Int. Cl. A61B 5/0215; A61B 5/021

(52) U.S. Cl. CPC A61B 5/02152; A61B 5/02141

(57) ABSTRACT

A device used to quickly measure central venous pressure is disclosed. Compared to current systems, the JVPM is easier to use, less expensive, and faster in providing the needed information to determine both the course of treatment and the proper positioning of the inserted catheter. As a physically smaller device that remains within the sterile operative field, the JVPM is also less likely to introduce infecting organisms into the body.

### RELATED APPLICATION

This application claims priority to U.S. Provisional Application No. 62/020,594, which was filed on Jul. 3, 2014 by the same inventors as the present application, and which is entitled “A Device Used to Quickly Measure Central Venous Pressure.” The aforementioned priority application is herein incorporated by reference for all that it teaches expressly and implicitly without exclusion of any part thereof.

### TECHNICAL FIELD

The present disclosure is related generally to checking a patient's central venous pressure, and, more particularly, to a system and method for quickly and accurately determining a patient's central venous pressure in an emergency situation.

### BACKGROUND

Currently, when a physician inserts a catheter into the central venous system of a patient, there is no device available to immediately determine pressure in the central venous system. The lack of immediate pressure information generally delays certain types of treatment. Moreover, current systems do not apprise the physician when the catheter has mistakenly been placed in an artery and not in a vein. A catheter that is positioned in an artery can cause serious bleeding and stroke in the patient.

The current methods are also generally difficult to use. One current method for measuring venous pressure involves the use of a manometer, which is slow and cumbersome to employ in a sterile field. Another method, the electronic pressure transducer, requires significant time and expertise to properly utilize and may introduce a non-sterile element into the operative field. Moreover, confirmation of proper catheter placement currently requires a chest radiograph or arterial blood gas analysis. Both of these methods are expensive and are slow to yield results.

While the present disclosure is directed to a system that can eliminate some of the shortcomings noted in this Background section, it should be appreciated that any such benefit is not a limitation on the scope of the disclosed principles, nor of the attached claims, except to the extent expressly noted in the claims. Additionally, the discussion of technology in this Background section is reflective of the inventors' own observations, considerations, and thoughts, and is in no way intended to accurately catalog or comprehensively summarize the prior art. As such, the inventors expressly disclaim this section as admitted or assumed prior art with respect to the discussed details. Moreover, the identification herein of a desirable course of action reflects the inventors' own observations and ideas, and should not be assumed to indicate an art-recognized desirability.

### BRIEF DESCRIPTION OF THE SEVERAL VIEWS OF THE DRAWINGS

While the appended claims set forth the features of the present techniques with particularity, these techniques, together with their objects and advantages, may be best understood from the following detailed description taken in conjunction with the accompanying drawings of which:

FIG. 1 is a cross-sectional schematic view of a device in accordance with an embodiment of the disclosed principles;

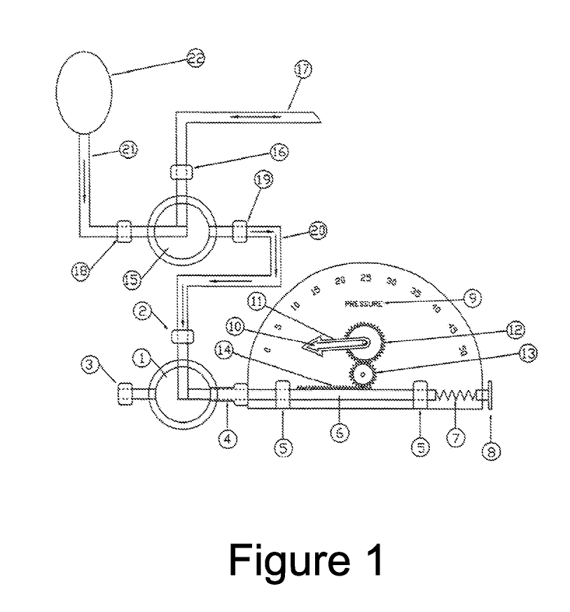

FIG. 2 is a cross-sectional schematic view of another device in accordance with an embodiment of the disclosed principles; and

FIG. 3 is a cross-sectional schematic view of a device in accordance with yet another embodiment of the disclosed principles.

### DETAILED DESCRIPTION

Before presenting a detailed discussion of embodiments of the disclosed principles, an overview of certain embodiments is given to aid the reader in understanding the later discussion. As noted above, currently, when a physician inserts a catheter into the central venous system there is no way to immediately determine pressure in the patient's central venous system. This means that an important piece of information needed to guide immediate treatment is missing.

There is also currently no device that can apprise the physician whether the catheter has been properly placed in a vein or has instead been misplaced in an artery. Those of skill in the art will appreciate that mispositioning a catheter in an artery can cause serious bleeding and stroke in the patient. The system described herein addresses these issues. The described system, referred to herein as the JVPM (Jackson Venous Pressure Manometer), allows the physician to make critical decisions concerning treatment more rapidly. In large part this is due to the ability of the JVPM to quickly determine the pressure in the central venous system.

The central venous pressure measurement guides decisions such as the administration of intravascular volume support during resuscitation for example. The JVPM also rapidly confirms the proper positioning of the catheter in a venous structure by confirming intraluminal pressure. In general, the current methods of measuring central venous pressure are cumbersome, potentially unsterile, time consuming and expensive. The JVPM provides pressure information rapidly, easily, and inexpensively and also improves and/or maintains sterility in the operative field.

In an embodiment of the disclosed principles, the JVPM system includes:
1. 3-port rotating monitoring valve.
2. Saline or body fluid charging communication port.
3. Waste Fluid flush communication port.
4. Piston retainer guide port.
5. Piston retainer guide.
6. Piston.
7. Piston calibration and tension spring.
8. Tension and calibration screw.
9. Jackson Pressure Manometer.
10. Peak Pressure Needle.
11. Secondary Pressure Needle.
12. Needlegear.
13. Piston gear.
14. Piston gear.
15. 3-port rotating valve.
16. Quick IV tubing connect.
17. Patient IV tubing.
18. Quick IV tubing connect.
19. Quick IV tubing connect.
20. Monometer IV tubing.
21. Flush IV tubing.
22. Flush reservoir.

Relationships Between The Components

The purpose of the JVPM is to determine internal physiologic peak and static pressures as an aid to diagnostic and treatment plans. Access is obtained by inserting a needle into the appropriate blood vessel or internal structural source to be monitored for pressure, and attaching the needle to IV tubing 17. The end of the IV tubing 17 is attached to the 3-port rotating valve 15 with Quick IV tubing connect 16. A pressurized bag of tubing flush solution is attached to the 3-port rotating valve 15 at 18.

The Manometer IV tubing 20 is attached to the 3-port rotating valve 15 at 19. The end of IV tubing 20 is attached to the saline or body fluid charging communication port 2 of the saline or body fluid charging communication port of the 3-port Rotating Monitoring Valve 1. The waste fluid flush communication port 3 is left capped until flushing the system is required. The Jackson Pressure Manometer 9 is connected to the 3-port Rotating Monitoring Valve 1 at the Piston retainer guide port 4. In use, the Jackson Pressure Monitor is placed in a level position with the Superior vena cava or body compartment to be measured.

As illustrated the JVPM is a small, easy-to-use, self-contained, sterile measuring device. The JVPM easily and securely fits into the closed tubing system that is customarily used with a central venous catheter or other body system pressure measuring conduit. The JVPM directly and immediately measures the selected physiologic pressure and does not require a power source. Both static and dynamic pressures are displayed (via the Peak Pressure Needle 10 and Secondary Pressure Needle 11). The JVPM maintains sterility in the operative field, immediately identifies improper positioning of the vascular catheter and also rapidly and easily determines the central venous pressure, guiding urgent treatment decisions.

If a catheter is malpositioned in an artery rather than a vein, the measured pressure easily reflects the mistake, since the arterial system is much higher pressure than the venous system. The measured central venous pressure may be used to guide decisions on resuscitating a critically ill patient, especially with respect to the administration of intravascular crystalloids and colloids or other medications. A low central venous pressure in a hypotensive patient favors the aggressive infusion of fluid while higher venous pressures suggest more cautious use of intravenous fluids that may aggravate breathing problems such as pulmonary edema.

For distribution, the JVPM may be offered in sterile fashion either in a commonly used sterile central venous pressure catheter placement kit, or in a separate, sterile package. As can be seen, the JVPM is a self-contained device that holds all the elements necessary for its function. The pressure monitoring readout may be configured to display peak, mean, systolic or diastolic pressure, or any combination of pressures. The JVPM can be used for short-term or long term clinical monitoring. The fluid tubing elements that attach to the JVPM may be configured in several ways, as demonstrated in the technical diagrams included with this application. Measurement of pressure by the JVPM could be determined by a spring-loaded system, gear system, diaphragmatic pressure sensor or piezoelectric device, or by other powered or unpowered sensors.

The JVPM provides an immediate measurement of pressure in the target blood vessel or other compartment. A low pressure in a blood vessel confirms that the catheter is properly placed in a central vein. A high pressure indicates improper positioning in an artery. Further, the specific pressure in the central vein guides immediate treatment decisions. A very low pressure might demand that more intravenous fluid be provided to the patient. A higher pressure, still within the venous pressure range, would argue against more intravenous fluid in order to spare the patient the adverse consequences of receiving too much fluid.

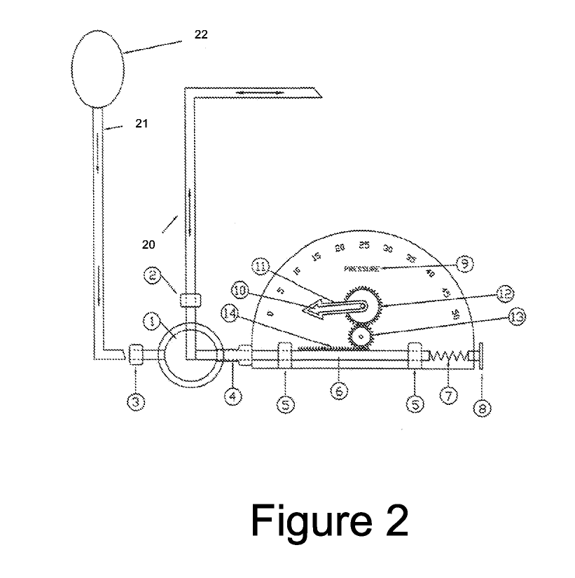

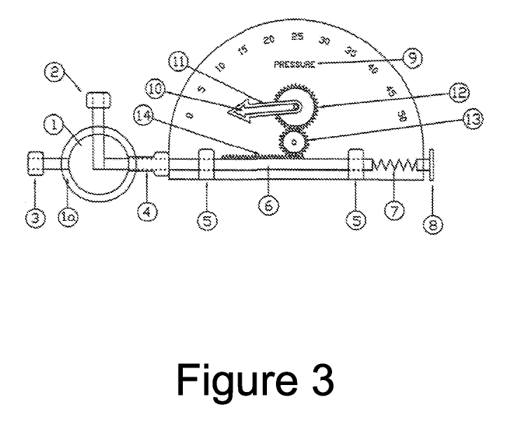

Other configurations of the system are shown in FIGS. 2 and 3.

It will be appreciated that various systems and processes for automated attendee data collection have been disclosed herein. However, in view of the many possible embodiments to which the principles of the present disclosure may be applied, it should be recognized that the embodiments described herein with respect to the drawing figures are meant to be illustrative only and should not be taken as limiting the scope of the claims. Therefore, the techniques as described herein contemplate all such embodiments as may come within the scope of the following claims and equivalents thereof.

We claim:

1. A rapid venous pressure monitoring system comprising a self-contained pressure gauge having an inlet valve attachable via tubing to a patient, the gauge including a dynamic and peak pressure reading mechanism.

2. The rapid venous pressure monitoring system in accordance with claim 1, further comprising a 3-port rotating monitoring valve, a saline or body fluid charging communication port and a waste fluid flush communication port.

3. The rapid venous pressure monitoring system in accordance with claim 1, further comprising a piston linked to a piston calibration and tension spring and tension and calibration screw.

4. The rapid venous pressure monitoring system in accordance with claim 1, wherein the dynamic and peak pressure reading mechanism comprises a peak pressure needle and a secondary pressure needle.

5. The rapid venous pressure monitoring system in accordance with claim 4, wherein the dynamic and peak pressure reading mechanism further comprises one or more needle gears and one or more piston gears.
